(19) United States
(12) Patent Application Publication
(10) Pub. No.: US 2025/0092142 A1
(43) Pub. Date: Mar. 20, 2025

(54) MONOCLONAL ANTIBODY TARGETING FZD7, PREPARATION METHOD AND USE THEREOF

(71) Applicant: SHANGHAITECH UNIVERSITY, Shanghai (CN)

(72) Inventors: Fei XU, Shanghai (CN); Lu XU, Shanghai (CN); Tingting WU, Wuxi (CN)

(21) Appl. No.: 18/470,595

(22) Filed: Sep. 20, 2023

(51) Int. Cl. C07K 16/28; A61K 47/68; C07K 14/725; C12N 15/85

(52) U.S. Cl. CPC C07K 16/2863; A61K 47/68031; A61K 47/6845; C07K 14/7051; C12N 15/85; C12N 2800/107

(57) ABSTRACT

The present disclosure discloses a monoclonal antibody targeting FZD7, preparation method and use thereof. The monoclonal antibody targeting FZD7 comprises a heavy chain variable region and a light chain variable region, the heavy chain variable region comprises an amino acid sequence of SEQ ID NO: 1 or an amino acid sequence with at least 99% sequence identity to sequence of SEQ ID NO: 1, the light chain variable region comprises an amino acid sequence of SEQ ID NO: 2 or an amino acid sequence having at least 99% sequence identity to sequence of SEQ ID NO: 2. The monoclonal antibody targeting FZD7 obtained by the present disclosure binds to the FZD7 protein both in vitro and in vivo and has clinical development value.

### SEQUENCE LISTING

This application contains references to amino acid sequences and/or nucleic acid sequences which have been submitted concurrently herewith as the sequence listing xml file entitled “000180us_SequenceListing.XML”, file size 7.0 KiloBytes (KB), created on Sep. 21, 2023. The aforementioned sequence listing is hereby incorporated by reference in its entirety pursuant to 37 C.F.R. § 1.52(e)(5).

### TECHNICAL FIELD

The present disclosure belongs to the field of biotechnology, and relates to a monoclonal antibody targeting FZD7, preparation method and use thereof, in particular to a highly efficient monoclonal antibody targeting FZD7, preparation method and use thereof; the present disclosure also provides a nucleic acid molecule encoding the antibody, an expression vector, a host cell, and the method for expressing the antibody.

### BACKGROUND

The Frizzleds (FZDs) receptor family is a member of the G protein-coupled receptor (GPCR) F family, with 10 isoforms that are widely expressed in the cardiovascular system, etc. The targeting role of FZDs in tumors has been reported.

Frizzled7 (FZD7) gene is localized on chromosome 2q33 and consists of 574 amino acids. It is highly expressed in melanoma, lung cancer, esophageal cancer, gastric cancer, colon cancer, liver cancer and lymphocytic leukemia (Khan N I et al., Br J Haematol, 2007, 138:338-348). Studies have confirmed that FZD7 is an essential biomarker in the development of many cancers (Yang L et al., Oncogene, 2011, 30(43): 4437-4446) and is one of the potential cancer therapeutic targets in vivo.

No FZDs-related drugs have been available on the market for the time being, and the most advanced drug candidate targeting FZDs in research worldwide is in clinical phase I, which is a multi-target drug vantictumab inhibiting wnt, FZDs and (3-catenin signaling, and no drug specifically targeting FZD7 is in research. Therefore, the development of monoclonal antibodies targeting FZD7 isoform of FZD target is necessary and has important medical value.

### CONTENT OF THE PRESENT DISCLOSURE

The technical problem to be solved by the present disclosure is to overcome the defect of the lack of effective antibody targeting FZD7 drug in the prior art, therefore providing a monoclonal antibody targeting FZD7, preparation method and use thereof.

The technical solutions of the present disclosure to solve the technical problem described above are as follows.

The first technical solution of the present disclosure is: A monoclonal antibody targeting FZD7, comprising a heavy chain variable region and a light chain variable region, the heavy chain variable region comprises an amino acid sequence of SEQ ID NO: 1 or an amino acid sequence with at least 99% sequence identity to sequence of SEQ ID NO: 1, the light chain variable region comprises an amino acid sequence of SEQ ID NO: 2 or an amino acid sequence with at least 99% sequence identity to sequence of SEQ ID NO: 2.

The antibody of the present disclosure can further comprise a heavy chain constant region and a light chain constant region; preferably, subtype of the heavy chain constant region is IgG1, and the light chain constant region is kappa chain.

In a preferred embodiment of the present disclosure, the monoclonal antibody targeting FZD7 targets a protein comprising an amino acid sequence of SEQ ID NO: 3.

The monoclonal antibody targeting FZD7 in the present disclosure which presents within full-length antibody, Fab, Fab′, F(ab′)₂, Fv, bispecific antibody or multi-specific antibody, i.e. it may be in the form of full-length antibody, Fab, Fab′, F(ab′)₂, Fv, bispecific antibody or multi-specific antibody.

In the present disclosure, term “full-length antibody” is used interchangeably to refer to a glycoprotein comprising at least two heavy chains (HC) and two light chains (LC) interconnected by disulfide bonds. Each heavy chain consists of a heavy chain variable region (abbreviated as VH in the present disclosure) and a heavy chain constant region. The heavy chain constant region consists of 3 domains of CH1, CH2 and CH3. Each light chain consists of a light chain variable region (abbreviated as VL in the present disclosure) and a light chain constant region (abbreviated as CL in the present disclosure). The light chain constant region consists of a domain of CL. Heavy chains of mammalian antibodies are classified as α, δ, ε, γ and μ. Light chains of mammalian antibodies are classified as λ or κ. Immunoglobulins having heavy chains α, δ, ε, γ and μ are classified as immunoglobulin (Ig) IgA, IgD, IgE, IgG and IgM. Complete antibody forms a “Y” shape. The stem of Y consists of the second and third constant regions (and for IgE and IgM, the fourth constant region) of the two heavy chains that are bound together, and disulfide bonds (inter-chain) are formed between hinges. Heavy chains γ, α and δ have a constant region composed of three tandem Ig domains (in one row) and a hinge region for added flexibility; heavy chains and F have a constant region composed of four immunoglobulin domains. The second and third constant regions are called “CH2 domain” and “CH3 domain”, respectively. Each arm of antibody comprises variable region and constant region of a single light chain and variable region and first constant region of a single heavy chain which are bound together. The variable region of the light chain and the variable region of the heavy chain are responsible for antigen binding.

In the present disclosure, “Fab fragment” consists of alight chain and CH1 and variable region of a heavy chain. Heavy chain of the Fab molecule cannot form a disulfide bond with another heavy chain molecule. “Fc” region is composed of two heavy chain fragments containing the CH2 and CH3 domains of an antibody. The two heavy chain fragments are combined together by two or more disulfide bonds and by the hydrophobic interaction between the CH3 structural domains. “Fab′ fragment” contains a light chain and a portion of a heavy chain containing the VH, CH1 domain and the region between the CH1 and CH2 domains, thereby the two Fab′ fragments can form an interchain disulfide bond between the two heavy chains of the two Fab′ fragments to form the F(ab′)₂ molecule. “F(ab′)₂ fragment” contains two light chains and two heavy chains containing portions of the constant region between the CH1 and CH2 domains, thereby forming an interchain disulfide bond between the two heavy chains. Thus, the F(ab′)₂ fragment consists of two Fab′ fragments bound together by disulfide bond between the two heavy chains. Term “Fv” refers to the antibody fragment consisting of the VL and VH domains of a single arm of the antibody, but lacking constant region.

In the present disclosure, the scFv (single chain antibody fragment) may be a conventional single chain antibody in the art comprising a heavy chain variable region, a light chain variable region and a short peptide of 15 to 20 amino acids. Wherein VL and VH domains pair together to form a monovalent molecule by a linker which enables them to be one single polypeptide chain [reference, e.g., Bird et al, Science 242:423-426 (1988) and Huston et al, Proc. Natl. Acad. Sci. USA 85:5879-5883 (1988)]. Such scFv molecules may have the general structure: NH₂—VL-linker-VH—COOH or NH₂-VH-linker-VL-COOH. Suitable linker in prior art consists of an amino acid sequence of repeated G₄S or a variant thereof. For example, a linker having an amino acid sequence of (G4S)₄ or (G4S)₃ may be used, but variants thereof may also be used.

Term “multi-specific antibody” is used in its broadest sense to cover antibodies with multi-epitope specificity. These multi-specific antibodies include but are not limited to: antibodies comprising a heavy chain variable region (VH) and a light chain variable region (VL), wherein the VH-VL unit has multi-epitope specificity; antibodies comprising two or more VL and VH regions, wherein each VH-VL unit binding to a different target or a different epitope of the same target; antibodies comprising two or more single variable regions, wherein each of single variable regions binds to a different target or a different epitope of the same target; full-length antibodies, antibody fragments, bispecific antibodies (diabodies), and triabodies, antibody fragments covalently or non-covalently linked together, etc.

In a preferred embodiment of the present disclosure, the preparation method of the monoclonal antibody targeting FZD7 comprises the following steps:
(1) An expression vector is constructed to express DNA sequence of SEQ ID NO: 4 to obtain FZD7 linker protein;
(2) Mice are immunized using the FZD7 linker protein obtained in step (1);
(3) Immunized mice are taken to prepare spleen cell suspension, which is then fused with SP2/0 myeloma cells to prepare hybridoma cells;
(4) Antibodies are prepared from ascites.

The second technical solution of the present disclosure is: An isolated nucleic acid encoding the monoclonal antibody targeting FZD7 of the first technical solution.

The third technical solution of the present disclosure is: A recombinant expression vector comprising the isolated nucleic acid of the second technical solution; preferably, the recombinant expression vector is plasmid, cosmid, phage or viral vector, the viral vector is preferably retroviral vector, lentiviral vector, adenoviral vector or adeno-associated viral vector; the recombinant expression vector preferably has a backbone of plasmid pET28a-Sumo.

In the present disclosure, term “host cells” may include cells into which exogenous nucleic acids have been introduced, including the progeny of these cells. Host cells include “transformants” and “transformed cells”, which include primary transformed cells as well as the progeny derived therefrom, regardless of the number of transgenerations. Progeny may not be identical to the parental cells in terms of nucleic acid content, and may contain mutations. The present disclosure includes mutant progeny that have the same function or biological activity as the cells screened or selected in the initial transformed cells.

The fourth technical solution of the present disclosure is: A transformant comprising the recombinant expression vector of the third technical solution in host cells.

As used in the present disclosure, “vector” represents a construct that is capable of delivering one or more genes or sequences of interest into a host cell and preferably expressing the genes or sequences in the host cell. Examples of vectors include, but not limited to viral vectors, naked DNA or RNA expression vectors, plasmid, cosmid or phage vectors, DNA or RNA expression vectors associated with cationic coagulants, DNA or RNA expression vectors capsulated in liposomes and certain eukaryotic cells, such as production cells.

The fifth technical solution of the present disclosure is: A chimeric antigen receptor comprising the monoclonal antibody targeting FZD7 of the first technical solution.

The sixth technical solution of the present disclosure is: A genetically modified cell comprising the chimeric antigen receptor of the fifth technical solution; preferably, the genetically modified cell is a eukaryotic cell, preferably an isolated human cell; more preferably an immune cell such as a T cell, or an NK cell.

The seventh technical solution of the present disclosure is: A preparation method of a monoclonal antibody targeting FZD7, which comprises culturing the transformant of the fourth technical solution and obtaining the monoclonal antibody targeting FZD7 from the culture.

The eighth technical solution of the present disclosure is: An antibody-drug conjugate comprising a cytotoxic agent, and the monoclonal antibody targeting FZD7 of the first technical solution; the cytotoxic agent is toxic molecule; preferably, the cytotoxic agent is MMAF or MMAE.

The ninth technical solution of the present disclosure is: A pharmaceutical composition comprising the monoclonal antibody targeting FZD7 of the first technical solution and/or the antibody-drug conjugate of the eighth technical solution, and a pharmaceutically acceptable carrier, the pharmaceutical composition is preferably a cancer-related therapeutic agent specifically targeting FZD7;
preferably, the pharmaceutical composition further comprises one or more of a group consisting of hormonal agents, targeted small molecule agents, proteasome inhibitors, imaging agents, diagnostic agents, chemotherapeutic agents, oncolytic agents, cytotoxic agents, cytokines, activators of co-stimulatory molecules, inhibitors of inhibitory molecules and vaccines.

In some embodiments, the pharmaceutical composition or pharmaceutical agent in the present disclosure comprises a suitable pharmaceutically acceptable carrier such as a pharmaceutical excipient, for example, a pharmaceutical carrier or a pharmaceutical excipient, including a buffering agent known in the art. As used in the present disclosure, “pharmaceutically acceptable carrier” or “pharmaceutical carrier” includes any and all physiologically compatible solvents, dispersion media, isotonic agents, absorption retardants, etc. Pharmaceutical carriers suitable for use in the present disclosure may be sterile liquids such as water and oils, including those of petroleum, animal, vegetable or synthetic origin, such as peanut oil, soybean oil, mineral oil, sesame oil, etc. Water is a preferred carrier when administering pharmaceutical composition intravenously. Saline solutions, aqueous dextrose and glycerol solutions can also be used as liquid carriers, especially for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, etc. For the use of excipients and their applications, “Handbook of PHarmaceutical Excipients” (5^{th} edition, R. C. Rowe, P. J. Seskey and S. C. Owen, PHarmaceutical Press, London, Chicago) can also be referred to. If desired, the composition may also contain a small amount of a humectant or emulsifier, or a pH buffer. These compositions may be in the form of solutions, suspensions, emulsions, tablets, pills, capsules, powders, sustained release formulations, etc. Oral formulations may contain standard pharmaceutical carriers and/or excipients such as pharmaceutical grade mannitol, lactose, starch, magnesium stearate and saccharin. Pharmaceutical agents or pharmaceutical compositions comprised the present disclosure described herein can be prepared by mixing the antibody or antigen-binding fragment thereof of the present disclosure having the desired purity with one or more optional pharmaceutical excipients (Remington's PHarmaceutical Sciences, 16^{th} edition, Osol, A., ed. (1980)), preferably in the form of lyophilized formulation or aqueous solution. The pharmaceutical compositions or agents of the present disclosure may also comprise more than one active ingredient, the active ingredient is necessary for the particular indication being treated, preferably those having complementary activities that do not adversely affect each other. For example, it is desirable of the pharmaceutical composition to also provide other anti-infective active ingredients, such as other antibodies, anti-infective agents, small molecule drugs or immunomodulators, etc. The active ingredients are present in suitable combinations with an effective amount for the intended use. Sustained release formulations may be prepared. Suitable examples of sustained release formulations include semi-permeable matrices containing solid hydrophobic polymers of the antibodies of the present disclosure or antigen-binding fragments thereof, the matrices are in the form of shaped articles, such as films or microcapsules.

The tenth technical solution of the present disclosure is: A use of the monoclonal antibody targeting FZD7 of the first technical solution, the antibody-drug conjugate of the eighth technical solution and/or the pharmaceutical composition of the ninth technical solution in the preparation of a drug for diagnosis, prevention and/or treatment of specifically targeted FZD7-associated cancer; preferably, the tumor is FZD7-positive tumor.

The present disclosure applies the following technical solutions:

An antibody that specifically binds to FZD7 has an amino acid sequence derived from mouse.

A preparation method of a monoclonal antibody targeting FZD7: Mice are immunized by the constructed pET28a-Sumo expression vector, the vector is transformed and expression is induced. After the serum titer of the mice exceed 1:10,000, the spleen of the mice is isolated to make spleen cell suspension, which is then fused with SP2/0 myeloma cells to prepare hybridoma cells, and ELISA screening and subcloning are performed to obtain a hybridoma cell line that stably secreted FZDT antibody. The screened hybridoma cells are injected into the peritoneal cavity of mice sensitized with paraffin oil, and ascites is taken from the mice before dying, and the antibody in the ascites is purified to be the monoclonal antibody targeting FZD7.

On the basis of common knowledge in the art, each of the preferred conditions described above can be combined arbitrarily to obtain each preferred example of the present disclosure.

The reagents and raw materials used in the present disclosure are commercially available.

The positive and progressive effects of the present disclosure are:

The immunogen used in the present disclosure to prepare the monoclonal antibody targeting FZD7 was synthesized in vitro using the gene of the immunogen, and after constructing the pET28a-Sumo expression vector, the recombinant was expressed and then the production was verified as the target protein by ELISA. The antibody preparation method is based on hybridoma cell technology to prepare monoclonal antibodies. Both in vitro SEC binding assay and validation of binding by Western blot verified that the monoclonal antibody targeting FZD7 obtained by the present disclosure could bind to FZD7 protein. Living cell binding assays also verified that the monoclonal antibody targeting FZD7 obtained by the present disclosure bound to FZD7 protein. It is presumed that the monoclonal antibody targeting FZD7 obtained by the present disclosure has clinical development value.

### BRIEF DESCRIPTION OF THE DRAWINGS

FIG. 1 is the plasmid profile of FZD7 DNA template.

FIG. 2 is the peak map of the analytical molecular sieve experiment.

FIG. 3 is the protein immunoblotting experiments to verify binding.

FIG. 4 shows binding experiments detected by Fluorescence activated Cell Sorting (FACS).

### DETAILED DESCRIPTION OF THE EMBODIMENT

The following examples further illustrate the present disclosure, but the present disclosure is not limited to the scope of the examples. Experimental methods for which specific conditions are not indicated in the following examples are selected according to conventional methods and conditions, or according to the commercial specification.

### Example 1 FZD7 Expression Construct

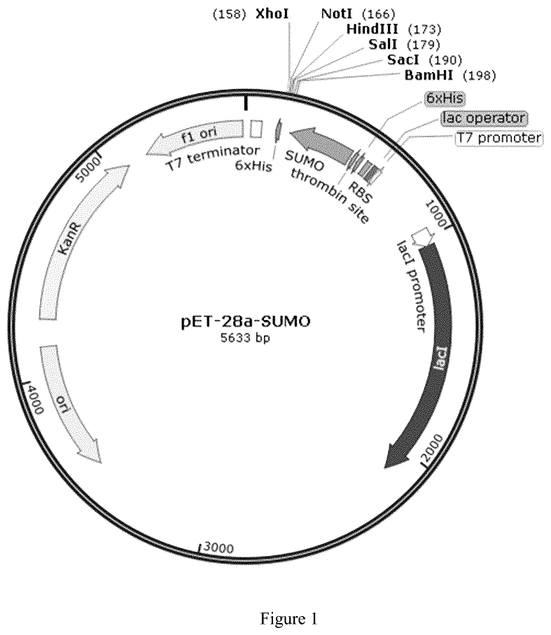

The pET28a-Sumo expression vector (FIG. 1) was constructed according to the expression gene of the target protein (SEQ ID NO: 4), and the FZD7 linker region protein was expressed.

### Example 2 Preparation of Hybridoma Cells

### 1. Animal Immunization

To obtain monoclonal antibodies targeting FZD7 linker protein, the expressed protein from Example 1 was selected by the present disclosure as the antigen to immunize mice (6-8 weeks BALB/c mice, Shanghai Sippe-Bk Lab Animal Co., Ltd.). Each mouse is immunized with 2 ml initial dose of an emulsifier made by mixing the immunized antigen with an equal amount of complete Freund's adjuvant by intraperitoneal injection. Boosters of an emulsifier made by mixing 1 ml of immune antigen with 1 ml of incomplete Freund's adjuvant were injected intraperitoneally at 2 week interval. One week after the third injection, blood was collected from the orbital vein of the mice, and the serum was collected for ELISA for detecting serum potency. Eligible mice immunized four times were selected for intraperitoneal injection with 40 μg of antigen, and cell fusion was performed three days later.

### 2. ELISA Test

1) Coating: antigen was diluted to 1 g/ml with CBS, and then added to the microplate, 100 μl per well, and the microplate was incubated overnight at 4° C.;
2) Blocking: coating solution was removed, 200 μl blocking buffer (5% skim milk powder) was added to each well, and was placed at 37° C. for 1.5 h;
3) Addition of sample: blocking buffer was removed, and sample was added, 100 μl per well, and was placed at 37° C. for 1 h;
4) Washing: the microplate was washed 3 times with wash buffer and then tap dried;
5) Secondary antibody: HRP-labeled goat anti-mouse was diluted to working concentration with blocking buffer, 100 μl per well, and was placed at 37° C. for 30 min;
6) Washing: the microplate was washed 3 times with wash buffer and then tap dried;
7) Chromogen: 100 μl TMB chromogen substrate was added to each well and was placed at 37° C. for 15 min;
8) Reaction stop: 50 μl stop solution was added to each well;
9) Readout: the plates were read for OD450 using a microplate reader.
3. Cell Fusion: Performed According to Standard Procedures in the Art.
3.1 SP2/0 cell collection:
3.1.1 50 ml SP2/0 cells in logarithmic growth phase and good condition was collected, and then centrifuged at 1200 rpm for 5 min;
3.1.2 The supernatant was removed, then 1 ml pre-heated fusion agent (PEG1450) was aspirated and added to the cells dropwise, and the mixture reacted in water bath at 37° C. for 1 min, and then stop solution was added (25 ml DMEM medium), followed by centrifuging at 1200 rpm for 5 min.
3.2 Fusion Plate Detection:
3.2.1 The growth status and contamination of the clones in the 96-well plates are observed every day after fusion to ensure normal cell growth;
3.2.2 Medium was replaced after the number of cells in the clonal cluster reached hundreds or more, 200 μl fresh HT medium was added to each well, then the plate was incubated in an incubator (37° C., 5% CO₂);
3.2.3 The medium was replaced after two days, 100 μl of supernatant was aspirated from each well to a coated 96-well microplate for ELISA test;
3.2.4 Positive wells containing cells were detected by ELISA, culture medium in the wells was removed and 200 μl fresh HT medium was added, followed by incubating the plate in an incubator (37° C., 5% CO2);
3.2.5 The medium was replaced after two days, and the wells were retested by ELISA. Positive wells were retested and prepared for subcloning.

### Example 3 Preparation of Monoclonal Antibody Targeting FZD7

1. Antibody Production in Murine Ascites
1.1 Paraffin oil was injected into the mice intraperitoneally 7 days in advance;
1.2 Cells were collected when the hybridoma cells basically covered the bottom of the bottle and the cells were in good condition, then the collected cells were centrifuged at 1200 rpm for 5 min and injected intraperitoneally into the paraffin-treated mice (2×10⁶ cells per mouse);
1.3 The status of mice was observed every day, the mice were executed by dislocation of cervical vertebra before death, the skin was cut with surgical scissor and torn to expose the peritoneum, and then a small cut was made in the peritoneum, and ascites were aspirated with a dropper;
1.4 Ascites was centrifuged at 3000 rpm for 30 min, the supernatant was aliquoted and frozen at −80° C., and a small amount of ascites was taken to detect the potency by ELISA.
2. Identification of Antibody Purity
2.1 1 ml ascites was taken for centrifugation at 12000 rpm for 4 min, the supernatant was collected and diluted with PBS;
2.2 1 ml column volume of proteinG column was equilibrated with 10 ml PBS, ascites sample was loaded and the flow-through was collected;
2.3 After washing with PBS, the column was eluted with 2.5 ml 0.1 M pH2.5 glycine solution, and the eluent was neutralized by adding 250 μl 1M Tris solution (pH8.8).
2.4 Antibody eluent was dialyzed overnight in PBS, and the affinity of antibody was detected by ELISA.

The reagents used for ELISA are as follows:
1) PBS (phosphate buffered saline) solution:
\textcircled{1} preparation method of PBS stock solution (10×):

| | |
|---|---|
| NaCl | 8.5 g |
| KCl | 0.2 g |
| Na₂HPO₄•12H₂O | 2.85 g (or Na₂HPO₄•2H₂O 1.13 g) |
| KH₂PO₄ | 0.27 g |

The components described above were dissolved in 100 ml deionized water and the pH value was adjusted to 7.2.
\textcircled{2} preparation method of PBS working solution (1×):

50 ml PBS stock solution was added with 450 ml deionized water to obtain the PBS working solution, then the solution could be stored at room temperature or 4° C. for later use.
2) Antigen coating solution (CBS, 1×):

1.59 g Na₂CO₃ and 2.93 g NaHCO₃ were added to 950 ml deionized water, and the pH value was adjusted to 9.6, the solution was added with deionized water to 1000 ml, and was then stored at 4° C.
3) Blocking buffer
5 g skim milk powder was dissolved in 100 ml PBS and mixed well, and the solution was then stored at 4° C. for later use (It is not suitable to store at 4° C. for more than three days.).
4) TMB chromogen solution
\textcircled{1} TMB stock solution: TMB powder was weighed to prepare 1.5 mg/ml of stock solution with DMSO, and the solution was then aliquoted and stored at −20° C.
\textcircled{2} NaAc buffer solution: 200 mM of NaAc solution was prepared and the pH value was adjusted by HAc to 5.3.
\textcircled{3} H₂O₂ solution: 0.03% of H₂O₂ solution was prepared.
\textcircled{4} Chromogen solution was prepared when used with a ratio of TMB stock solution:NaAc buffer solution:H₂O₂ solution=1:4:5, compound when it is in need.
5) Stop solution (2M of sulfuric acid)

100 ml of concentrated sulfuric acid was slowly added to 900 ml water (note: concentrated sulfuric acid must be added to water dropwise), and the solution was stored at room temperature for later use

Detailed procedures are as follows:
1) Coating: antigen was diluted with CBS, generally the concentration was 1 g/ml, and then the diluted antigen was added to the microplate at 100 μl per well, and the microplate was placed overnight at 4° C.
2) Blocking: coating solution was removed, then 200 μl of blocking buffer was added to each well, and the microplate was placed at 37° C. for 1.5 h.
3) Addition of sample: the blocking buffer was removed, and samples (diluted serum or primary antibody) were added at 100 μl per well to the microplate, and the microplate was placed at 37° C. for 1 h
4) Washing: the microplate was washed 3 times with tap water and then tap dry.
5) Addition of secondary antibody: secondary antibody was diluted to working concentration with the blocking buffer and added at 100 μl per well to the microplate, and the microplate was placed at 37° C. for 30 min.
6) Washing: the microplate was washed 10 times with tap water and then tap dry.
7) Addition of chromogen substrate: 100 μl per well of chromogen substrate was added to the microplate, and the microplate was placed at 37° C. for 15 min.
8) Addition of blocking buffer: 2 M 50 μl H₂SO₄ was added to each well.
9) Readout: plates were read for OD450 using a microplate reader.
   Report of Antibody ELISA data

### (1) Fusion Mouse ELISA Data (Assay Results of Mouse Immunized Three Times)

TABLE 1

| | Dilution multiple | | | | | | | | |
|---|---|---|---|---|---|---|---|---|---|
| OD_{450 nm} | | | | | | | | Negative | |
| Antibody name | 1250 | 2500 | 5000 | 10000 | 20000 | 40000 | 80000 | control | antigen |
| 3301-M1 | 2.017 | 2.124 | 2.218 | 2.169 | 1.825 | 1.127 | 0.715 | 0.148 | 3301-2K |
| 3301-M2 | 2.148 | 2.152 | 2.254 | 2.001 | 1.572 | 0.885 | 0.578 | 0.143 | (1 μg/ml) |
| 3301-M3 | 2.142 | 2.152 | 2.326 | 2.195 | 1.871 | 1.173 | 0.75 | 0.127 | |
| 3301-M4 | 2.194 | 2.057 | 2.264 | 1.952 | 1.437 | 0.826 | 0.491 | 0.142 | |
| 3301-M5 | 2.148 | 2.057 | 2.177 | 2.084 | 1.655 | 0.819 | 0.488 | 0.093 | |
| 3301-M6 | 2.154 | 2.139 | 2.091 | 1.868 | 1.353 | 0.706 | 0.408 | 0.065 | |

ELISA shows that the two groups of antibodies, 3301-M1 and 3301-M3, have the highest detection values. Therefore, spleen cells from these two groups of mice are selected to be fused with SP2/0 cells to prepare hybridoma cells.

The following seven monoclonal antibodies were prepared.

### (2) Results of ELISA for Ascites Antibodies

TABLE 2

| | | | | | | | | Negative | |
|---|---|---|---|---|---|---|---|---|---|
| Antibody name | 1250 | 2500 | 5000 | 10000 | 20000 | 40000 | 80000 | control | antigen |
| 3301-01 monoclonal | 2.894 | 2.785 | 2.787 | 2.847 | 2.848 | 2.785 | 2.821 | 0.096 | 3301-2K |
| antibody | | | | | | | | | (1 μg/ml) |
| 3301-02 monoclonal | 2.8 | 2.704 | 2.717 | 2.689 | 2.746 | 2.647 | 2.084 | 0.065 | |
| antibody | | | | | | | | | |
| 3301-03 monoclonal | 2.814 | 2.755 | 2.755 | 2.759 | 2.774 | 2.728 | 2.504 | 0.077 | |
| antibody | | | | | | | | | |
| 3301-04 monoclonal | 2.774 | 2.658 | 2.661 | 2.327 | 1.931 | 1.347 | 0.673 | 0.115 | |
| antibody | | | | | | | | | |
| 3301-05 monoclonal | 2.879 | 2.785 | 2.787 | 2.805 | 2.788 | 2.772 | 2.711 | 0.084 | |
| antibody | | | | | | | | | |
| 3301-06 monoclonal | 2.85 | 2.746 | 2.801 | 2.568 | 1.902 | 1.157 | 0.614 | 0.074 | |
| antibody | | | | | | | | | |
| 3301-07 monoclonal | 2.85 | 2.799 | 2.801 | 2.819 | 2.848 | 2.772 | 2.756 | 0.1 | |
| antibody | | | | | | | | | |

### Example 4 In Vitro Binding Assay of Antibody

### 1. Experiment of Analytic Size-Exclusive Chromography

1) Washing of membrane: in the cell homogenizer, low salt and high salt solution containing protease inhibitor (cocktail, bimake) was added respectively to wash the membrane, and the mixture was centrifuged at 35 000 rpm to retain the precipitate.

10× low salt solution (pH 7.4): 1 L solution containing 100 ml 1 M HEPES, 20.3 g MgCl₂·6H₂O, 14.9 g KCl.

1× high salt solution (pH 7.4): 5 L solution containing 50 ml 1 M HEPES, 10.165 g MgCl₂ 6H₂O, 7.45 g KCl, 292.5 g NaCl.

2) Lysis of membrane: after the precipitate was resuspended with low salt solution homogenate, 20 mg/ml iodoacetamide was added and incubated with lysing membrane solution for 2 h at 4° C.

2× Lysis buffer for membrane: 1 M HEPES, 5 M NaCl, 2% DDM, 0.4% CHS.

3) Affinity chromatography: centrifuge was performed at 35000 rpm to take the supernatant, cobalt medium (Co²⁺ resin) and 20 mM imidazole were added, and then incubated overnight at 4° C.

4) Purification: cobalt medium with membrane proteins was transferred to the purification column, W1 buffer and W2 buffer were added in turn by gravity to remove the heteroproteins that were not specifically bound to the cobalt medium. Finally, the membrane proteins were eluted from the cobalt medium with elute buffer and collected into a centrifuge tube for later use.

W1 buffer: 50 mM HEPES pH 7.4, 500 mM NaCl, 10% glycerol, 0.1% DDM, 0.02% CHS, 20 mM imidazole;

W2 buffer: 50 mM HEPES pH 7.4, 500 mM NaCl, 10% glycerol, 0.005% DDM, 0.001% CHS, 50 mM imidazole;

Elute buffer: 50 mM HEPES pH 7.4, 500 mM NaCl, 10% glycerol, 0.025% DDM, 0.005% CHS, 50 mM imidazole;

Membrane proteins were expressed by insect expression system, FZD7 membrane protein was obtained by steps of washing of membrane, lysis of membrane, affinity chromatography and purification, etc. 30 μg FZD7 protein was incubated with 120 μg of 5 #antibody (3301-05 monoclonal antibody) overnight at 4° C. in the refrigerator. A nanofilm SEC-250 column (sepax) was used to examine the protein that contains aromatic amino acids having a feature of absorption peak at UV 280 nm. Binding experiments were performed using an Agilent Technology 1260 Infinity instrument to detect the binding of FZD7 membrane protein and 5 #antibody (3301-05 monoclonal antibody).

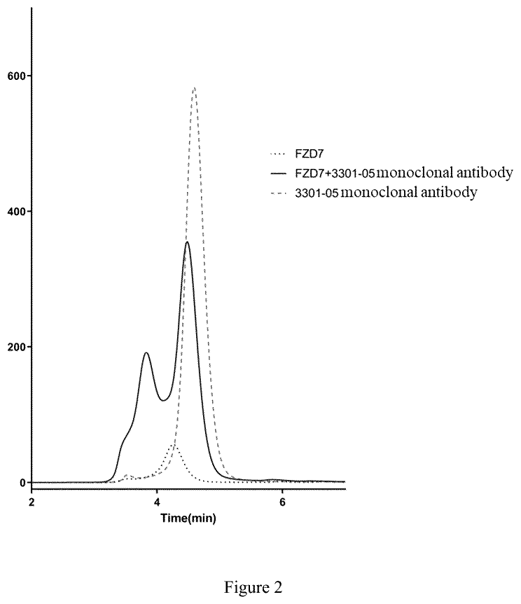

Result of analytic size-exclusive chromatography (aSEC) in FIG. 2 shows that at the same amount of FZD7 membrane protein, the curve of FZD7 membrane protein binding to 5 #antibody (3301-05 monoclonal antibody) has a clear peak shift compared to the curve of FZD7 membrane protein alone. It demonstrates that antibody 5 #(3301-05 monoclonal antibody) can bind to FZD7 membrane protein in vitro.

### 2. Protein Immunoblotting Experiments (Western Blot, WB)

Primary antibody: antibody 5 #(3301-05 monoclonal antibody)

Secondary antibody: Monoclonal ANTI-FLAG® M2 antibody produced in mouse 1 mg/mL, clone M2, affinity isolated antibody, buffered aqueous solution (Sigma)

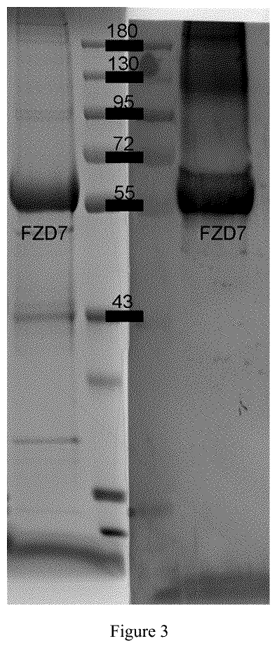

FZD7 membrane protein was extracted, and after SDS polyacrylamide gel electrophoresis (Sodium dodecyl sulfate-polyacrylamide gel electrophoresis, SDS-PAGE), the FZD7 membrane protein was electro transferred to membrane (PVDF membrane), and the membrane was blocked with TBST solution containing 5% milk. After incubated with primary antibody, the membrane was washed (TBST solution). Then, after incubated with secondary antibody, the membrane was washed (TBST solution) and stained by HRP, which can verify the binding of antibody to FZD7 membrane protein (FIG. 3) and show that antibody #5 (3301-05 monoclonal antibody) can effectively bind to FZD7 membrane protein.

### 3. Fluorescence Activated Cell Sorting (FACS) Experiment

10×10⁶ 293F/293T cells transiently transfected with FZD7 receptor (purchased from ATCC) were collected. After incubated with 5 #antibody (3301-05 monoclonal antibody) as primary antibody for 30 min, the cells were washed and followed by incubating for 30 min with secondary antibody (A31571 Alexa Fluor® 647 Donkey Anti-Mouse IgG (H+L), 1:2000), then washed and assayed on the machine (Millipore Guava). The negative control was cells only added with the secondary antibody.

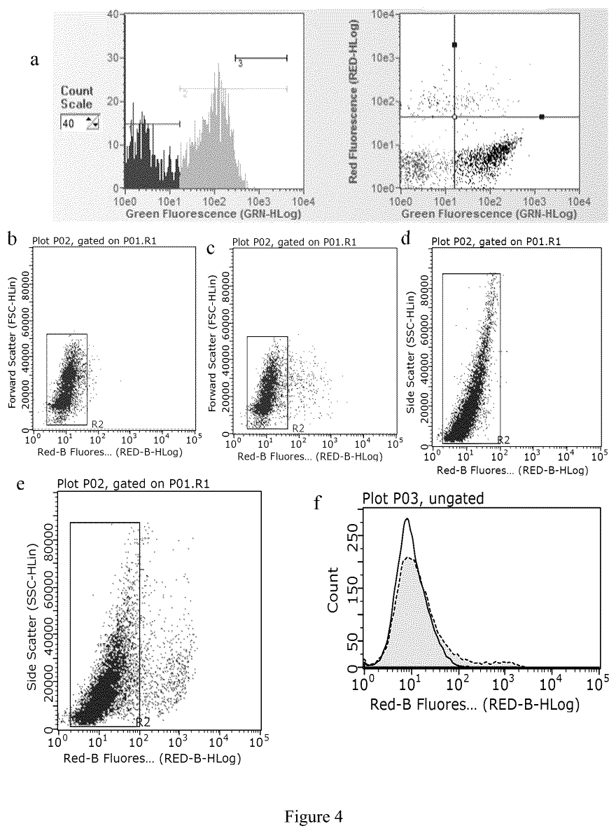

Groups of parts a, b and c in FIG. 4 are all 293F cells transiently transfected with FZD7 plasmid by Polyethylenimine, Linear (PEI, Polysciences). Wherein, part a shows a transfection efficiency of 63.6%. Cells are visible at the stronger red fluorescence (647) after the addition of 5 #antibody (3301-05 monoclonal antibody) and secondary antibody compared to control of part b, to which only secondary antibody was added. It implies that 293F live cells transiently transfected and expressing FZD7 can be bound by 5 #antibody (3301-05 monoclonal antibody) (part c in FIG. 4).

Groups of parts d and e in FIG. 4 are all 293T cells transiently transfected with FZD7 plasmids by Lipofectamine 2000 (ThermoFisher). Compared to control of part d, 293T live cells transiently transfected and expressing FZD7 could be bound by 5 #antibody (3301-05 monoclonal antibody) (e in FIG. 4) with a binding efficiency of 6.5% (part f in FIG. 4, a ratio of 594 cells/9132 cells).

What is claimed is:

1. A monoclonal antibody targeting FZD7, comprising a heavy chain variable region and a light chain variable region, the heavy chain variable region comprises an amino acid sequence of SEQ ID NO: 1 or an amino acid sequence with at least 99% sequence identity to sequence of SEQ ID NO: 1, the light chain variable region comprises an amino acid sequence of SEQ ID NO: 2 or an amino acid sequence with at least 99% sequence identity to sequence of SEQ ID NO: 2.

2. The monoclonal antibody targeting FZD7 of claim 1, wherein the monoclonal antibody targeting FZD7 comprises a heavy chain constant region and a light chain constant region; subtype of the heavy chain constant region is IgG1, the light chain constant region is kappa chain.

3. The monoclonal antibody targeting FZD7 of claim 1, wherein the monoclonal antibody targeting FZD7 targets a target protein comprising an amino acid sequence of SEQ ID NO: 3.

4. The monoclonal antibody targeting FZD7 of claim 1, which presents within full-length antibody, Fab, Fab′, F(ab′)₂, Fv, bispecific antibody or multispecific antibody.

5. The monoclonal antibody targeting FZD7 of claim 1, which has a preparation method comprising the following steps:
(1) An expression vector is constructed to express DNA sequence of SEQ ID NO: 4 to obtain FZD7 linker protein;
(2) Mice are immunized using the FZD7 linker protein obtained in step (1);
(3) Immunized mice are taken to prepare spleen cell suspension, which is then fused with SP2/0 myeloma cells to prepare hybridoma cells;
(4) Antibodies are prepared from ascites.

6. An isolated nucleic acid encoding the monoclonal antibody targeting FZD7 of claim 1.

7. A recombinant expression vector comprising the isolated nucleic acid of claim 6; preferably, the recombinant expression vector is plasmid, cosmid, phage or viral vector, the viral vector is preferably retroviral vector, lentiviral vector, adenoviral vector or adeno-associated viral vector; the recombinant expression vector preferably has a backbone of plasmid pET28a-Sumo.

8. A transformant comprising the recombinant expression vector of claim 7 in host cells.

9. A chimeric antigen receptor comprising the monoclonal antibody targeting FZD7 of claim 1.

10. A genetically modified cell comprising the chimeric antigen receptor of claim 9; preferably, the genetically modified cell is a eukaryotic cell, preferably an isolated human cell; more preferably an immune cell such as a T cell, or an NK cell.

11. A preparation method of a monoclonal antibody targeting FZD7, which comprises culturing the transformant of claim 8 and obtaining the monoclonal antibody targeting FZD7 from the culture.

12. An antibody-drug conjugate comprising a cytotoxic agent, and the monoclonal antibody targeting FZD7 of claim 1; the cytotoxic agent is toxic molecule; preferably, the cytotoxic agent is MMAF or MMAE.

13. A pharmaceutical composition comprising the monoclonal antibody targeting FZD7 of claim 1, and a pharmaceutically acceptable carrier, the pharmaceutical composition is preferably a cancer-related therapeutic agent specifically targeting FZD7;
preferably, the pharmaceutical composition further comprises one or more of a group consisting of hormonal agents, targeted small molecule agents, proteasome inhibitors, imaging agents, diagnostic agents, chemotherapeutic agents, oncolytic agents, cytotoxic agents, cytokines, activators of co-stimulatory molecules, inhibitors of inhibitory molecules and vaccines.

14. A pharmaceutical composition comprising the antibody-drug conjugate of claim 12, and a pharmaceutically acceptable carrier, the pharmaceutical composition is preferably a cancer-related therapeutic agent specifically targeting FZD7;
preferably, the pharmaceutical composition further comprises one or more of a group consisting of hormonal agents, targeted small molecule agents, proteasome inhibitors, imaging agents, diagnostic agents, chemotherapeutic agents, oncolytic agents, cytotoxic agents, cytokines, activators of co-stimulatory molecules, inhibitors of inhibitory molecules and vaccines.

15. A method for diagnosing, preventing or treating specifically targeted FZD7-associated tumor, which comprising administrating the monoclonal antibody targeting FZD7 of claim 1 to a subject in need thereof; preferably, the tumor is FZD7-positive tumor.

16. A method for diagnosing, preventing or treating specifically targeted FZD7-associated tumor, which comprising administrating the antibody-drug conjugate of claim 12 to a subject in need thereof, preferably, the tumor is FZD7-positive tumor.

17. A method for diagnosing, preventing or treating specifically targeted FZD7-associated tumor, which comprising administrating the pharmaceutical composition of claim 13 to a subject in need thereof; preferably, the tumor is FZD7-positive tumor.


SEQUENCE LISTING (5 sequences)

SEQ ID NO: 1 - protein, 120 aa, Mus musculus
QVQLLQSGPELMKPGTSVRMSCKTSGYTFSGYWMEWVKQRPGHGLEWIGEILPGTDKTNYNKNFKDKATLTADTSSSTAYLQVTSLTSEDSAVYFCVRGYGDSGSWFLYWGQGTLVTVST

SEQ ID NO: 2 - protein, 112 aa, Mus musculus
NIVMTQSPSSLAVSAGEKVTMSCKSSQSVLYSSNQKNCLAWYQQKPGQSPKLLIYWASTRESGVPDRFTGSGSGTDFTLTISSVQPEDLAVYYCHQYLSSWTFGGGTKLEIK

SEQ ID NO: 3 - protein, 74 aa, Mus musculus
DGSGGPGGGPTAYPTAPYLPDLPFTALPPGASDGRGRPAFPFSCPRQLKVPPYLGYRFLGERDCGAPCEPGRAN

SEQ ID NO: 4 - DNA, 222 bp, Mus musculus
  source 1..222: mol_type = other DNA
gacggctccggcggccccggcggcggccccaccgcctaccccacagccccttacctccctgatctgcctttcaccgccctgcctcccggagctagcgatggcagaggcagacctgccttccccttcagctgccccagacagctgaaggtgcccccctatctgggctacaggttcctgggcgagagagattgcggcgctccctgcgaacctggaagagccaac

SEQ ID NO: 5 - DNA, 1035 bp, Mus musculus
  misc_difference 1..3: note = n is a, c, g, or t
  source 1..1035: mol_type = other DNA
nnnagtggtggctacgtatactccggaatattaatagatcatggagataattaaaatgataaccatctcgcaaataaataagtattttactgttttcgtaacagttttgtaataaaaaaacctataaatattccggattattcataccgccccaccatcgggcgcgcggaattcgccaaaatgaagacaatcatcgccctgagctatatcttctgcctggtgttcgccgactacaaggacgacgacgacaaggaaaagggcattagcgtccccgaccacggcttctgtcagcccattagcatccccctgtgcaccgacatcgcctacaatcaaaccatcctgcccaacctcctgggccataccaaccaggaagacgctggactcgaagtccaccagttctaccccctggtgaaggtccagtgcagccccgagctgaggttcttcctctgctccatgtatgcccccgtgtgcaccgtgctggatcaggccatccccccctgtagaagcctctgtgaaagggccaggcagggatgcgaggctctgatgaacaagttcggcttccagtggcccgagaggctgaggtgtgagaacttccccgtgcacggcgccggcgaaatttgcgtgggacagaacacatccgacggctccggcggccccggcggcggccccaccgcctaccccacagccccttacctccctgatctgcctttcaccgccctgcctcccggagctagcgatggcagaggcagacctgccttccccttcagctgccccagacagctgaaggtgcccccctatctgggctacaggttcctgggcgagagagattgcggcgctccctgcgaacctggaagagccaacggcctcatgtacttcaaggaggaggagaggaggttcgccaggctctgggtgggagtgtggtccgtgctgtgctgcgcctccacactcttcaccgtgctgacctacctcgtggacatgaggagattcagctaccccgaaagacccatcatcttcctgtccggctgctatttcatg